(19) United States
(12) Patent Application Publication
(10) Pub. No.: US 2015/0085970 A1
(43) Pub. Date: Mar. 26, 2015

(54) SYSTEMS AND METHODS FOR HYBRID SCANNING

(71) Applicant: General Electric Company, Schenectady, NY (US)

(72) Inventors: Jean-Paul Bouhnik, Tirat Carmel (IL); Yulim Zingerman, Tirat Carmel (IL)

(73) Assignee: General Electric Company, Schenectady, NY (US)

(21) Appl. No.: 14/033,948

(22) Filed: Sep. 23, 2013

(51) Int. Cl. A61B 6/00

(52) U.S. Cl. CPC A61B 6/4241; A61B 6/502; A61B 6/4085
USPC 378/5; 378/19

(57) ABSTRACT

A system includes a detector and a processing unit. The detector includes multiple pixels configured to detect computed tomography (CT) events and nuclear medicine (NM) imaging events. The CT events correspond to X-rays emitted from a X-ray source through an object to be imaged, and the NM imaging events correspond to gamma rays emitted from a radiopharmaceutical that has been administered to the object. The detector is configured for photon counting detection of the CT events and the NM imaging events. The processing unit includes at least one processor and at least one memory comprising a tangible and non-transitory computer readable storage medium. The processing unit is configured to, based on corresponding energy levels of the CT events and the NM imaging events, identify CT information corresponding to the CT events and identify NM information corresponding to the NM imaging events.

### BACKGROUND OF THE INVENTION

The subject matter disclosed herein relates generally to imaging systems and techniques, and more particularly to hybrid scanning using plural scanning modalities.

Detectors for diagnostic imaging systems, for example, detectors for single photon emission computed tomography (SPECT) and computed tomography (CT) imaging systems are often produced from semiconductor materials, such as Cadmium Zinc Telluride (CdZnTe or CZT), Cadmium Telluride (CdTe) and Silicon (Si), among others. These semiconductor detectors typically include arrays of pixelated detector modules.

In some situations, it may be desirable to obtain or utilize information from more than one modality, for example, using SPECT and CT information. Conventional systems, however, may require the use of different detectors for different modalities and/or movement or different positioning of detectors relative to an object being imaged for different modalities. Such designs may result in increased time of scanning, expense of scanning, inconvenience of scanning, and/or patient discomfort or exposure. Known systems may also be inflexible and/or present shortcomings, difficulties, or drawback relating to registration of images generated using different imaging modalities.

### BRIEF DESCRIPTION OF THE INVENTION

In accordance with various embodiments, a system is provided including a detector and a processing unit. The detector includes multiple pixels configured to detect computed tomography (CT) events and nuclear medicine (NM) imaging events. The CT events correspond to X-rays emitted from a X-ray source through an object to be imaged, and the NM imaging events correspond to gamma rays emitted from a radiopharmaceutical that has been administered to the object. The detector is configured for photon counting detection of the CT events and the NM imaging events. For example, as used herein, a photon counting detector may be understood as a detector used to count individual photons. The detector and the X-ray source may be configured to rotate about the object to be imaged. The system may also include an asymmetric cone beam collimator disposed proximate the object and configured to be focused on the X-ray source, with the asymmetric cone beam collimator disposed at a shorter distance from the object than a distance from the X-ray source to the object. The processing unit includes at least one processor and at least one memory comprising a tangible and non-transitory computer readable storage medium. The processing unit is configured to, based on corresponding energy levels of the CT events and the NM imaging events, identify CT information corresponding to the CT events and identify NM information corresponding to the NM imaging events.

In accordance with various embodiments, a method is provided that includes detecting, with a detector comprising multiple pixels, computed tomography (CT) events and nuclear medicine (NM) imaging events. The CT events correspond to X-rays emitted from a X-ray source through an object to be imaged, and the NM imaging events correspond to gamma rays emitted from a radiopharmaceutical that has been administered to the object. The detector and the X-ray source, as well as a cone beam collimator disposed proximate the object and associated with the detector, wherein the cone beam collimator is substantially closer to the object than the X-ray source, may be rotated about the object during the detecting. The method also includes identifying, based on energy levels of the CT events, CT information corresponding to the CT events. Further, the method includes identifying, based on energy levels of the NM imaging events, NM information corresponding to the NM imaging events.

In accordance with various embodiments, a tangible and non-transitory computer readable medium is provided. The tangible and non-transitory computer readable medium includes one or more computer software modules configured to direct one or more processors to obtain, from a detector comprising multiple pixels, imaging information corresponding to computed tomography (CT) events and nuclear medicine (NM) imaging events. The CT events correspond to X-rays emitted from a X-ray source through an object to be imaged. The NM imaging events correspond to gamma rays emitted from a radiopharmaceutical that has been administered to the object. The one or more computer software modules are also configured to direct one or more processors to identify, based on energy levels of the CT events, CT information corresponding to the CT events. Also, the one or more computer software modules are configured to direct one or more processors to identify, based on energy levels of the NM imaging events, NM information corresponding to the NM imaging events.

### BRIEF DESCRIPTION OF THE DRAWINGS

FIG. 1 is a schematic diagram of an imaging system in accordance with various embodiments.

FIG. 2 is a graph showing energy windows in accordance with various embodiments.

FIG. 3 is a schematic view of an imaging system in accordance with various embodiments.

FIG. 4 is a flowchart of a method for imaging in accordance with various embodiments.

FIG. 5 is a top perspective view of a pixelated photon detector formed in accordance with various embodiments.

FIG. 6 is a top perspective view of a gamma camera including a plurality of pixelated photon detectors in accordance with various embodiments.

FIG. 7 is a perspective view of an exemplary nuclear medicine imaging system constructed in accordance with various embodiments.

FIG. 8 is a block diagram of a nuclear medicine imaging system constructed in accordance with various embodiments.

### DETAILED DESCRIPTION OF THE INVENTION

The following detailed description of certain embodiments will be better understood when read in conjunction with the appended drawings. To the extent that the figures illustrate diagrams of the functional blocks of various embodiments, the functional blocks are not necessarily indicative of the division between hardware circuitry. Thus, for example, one or more of the functional blocks (e.g., processors or memories) may be implemented in a single piece of hardware (e.g., a general purpose signal processor or random access memory, hard disk, or the like) or multiple pieces of hardware. Similarly, the programs may be stand-alone programs, may be incorporated as subroutines in an operating system, may be functions in an installed software package, and the like. It should be understood that the various embodiments are not limited to the arrangements and instrumentality shown in the drawings.

As used herein, the terms “system,” “unit,” or “module” may include a hardware and/or software system that operates to perform one or more functions. For example, a module, unit, or system may include a computer processor, controller, or other logic-based device that performs operations based on instructions stored on a tangible and non-transitory computer readable storage medium, such as a computer memory. Alternatively, a module, unit, or system may include a hard-wired device that performs operations based on hard-wired logic of the device. Various modules or units shown in the attached figures may represent the hardware that operates based on software or hardwired instructions, the software that directs hardware to perform the operations, or a combination thereof.

“Systems,” “units,” or “modules” may include or represent hardware and associated instructions (e.g., software stored on a tangible and non-transitory computer readable storage medium, such as a computer hard drive, ROM, RAM, or the like) that perform one or more operations described herein. The hardware may include electronic circuits that include and/or are connected to one or more logic-based devices, such as microprocessors, processors, controllers, or the like. These devices may be off-the-shelf devices that are appropriately programmed or instructed to perform operations described herein from the instructions described above. Additionally or alternatively, one or more of these devices may be hard-wired with logic circuits to perform these operations.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Also as used herein, the phrase “image” or similar terminology is not intended to exclude embodiments in which data representing an image is generated, but a viewable image is not. Therefore, as used herein the term “image” broadly refers to both viewable images and data representing a viewable image. However, certain embodiments generate, or are configured to generate, at least one viewable image.

Various embodiments provide systems and methods for hybrid scanning (e.g., imaging with two or more modalities using a single type of detector). Various embodiments provide for concurrent or simultaneous acquisition of physiological and anatomical data. For example, in various embodiments, computed tomography (CT) imaging information and nuclear medicine (NM) imaging information (e.g., single photon emission computed tomography (SPECT)) may be acquired simultaneously or concurrently. A common detector or detectors may be used to acquire both CT and NM information. The common detector or detectors may be configured to detect gamma rays and X-rays, and may be configured for photon counting CT. In some embodiments, a SPECT/CT hybrid imaging system may be used in conjunction with molecular breast imaging (MBI).

Detected information may be grouped by energy levels. For example, one energy window or bin may be used in connection with NM data, and a different energy window or bin may be used in connection with CT data, with the NM data and the CT data collected with or acquired via the same detector, and with the same detector in the same position relative to the object to be imaged. Thus, detected events caused by X-ray transmission from the X-ray source may be identified based on the energy of the detected events and stored in a CT data grouping. For example, a CT energy window may be defined by a range of energies corresponding to X-rays from the X-ray source. All detected events (e.g., incidence of an X-ray upon one or more pixels) having an energy falling within the CT energy window may be identified as CT events and information regarding the CT events may be grouped together. Similarly, a NM energy window may be defined by a range of energies corresponding to gamma rays from an administered radiopharmaceutical. All detected events (e.g., incidence of a gamma ray upon one or more pixels) having an energy falling within the NM energy window may be identified as NM events and information regarding the NM events may be grouped together. Further still, in some embodiments, plural energy windows or bins may be used to sub-divide information from a particular type of scanning technique. For example, a first energy window may be used for NM data, a second energy window may be used for higher energy CT data, and a third energy window may be used for lower energy CT data.

Generally, in various embodiments, gamma and X-rays are acquired on the same detector simultaneously or concurrently (e.g., having at least a partial overlap between the times of acquisition of gamma rays and X-rays), and grouped into two or more energy bins or levels. It may be noted that, in some embodiments, the gamma rays and X-rays may be acquired and/or grouped consecutively or at different (e.g., non-overlapping) times, in contrast to simultaneously or concurrently. Because conventional CT imaging may provide a substantially higher X-ray flux than the event rate corresponding to NM imaging techniques, a X-ray source in some embodiments may have a substantially lower flux than conventional X-ray sources to avoid pile-up (e.g., the striking of plural photons on a detector in the same position between two readouts) and to permit use of an acquisition time appropriate for NM imaging without subjecting a patient to excessively or unnecessarily large doses of radiation from the X-ray source.

As indicated above, in some embodiments, one or more additional energy windows around a CT spectrum may be employed. The use of multiple CT energy windows may permit material decomposition, for example, to better identify and/or analyze calcification. For example, using a CT system or subsystem of about 120 kVp and a Tantalum filter, a double peak CT spectra may be achieved. A first peak may occur at about 60 keV and a second peak may occur about 90 keV. A first CT energy bin may be centered or oriented around the first peak and a second CT energy bin may be centered or oriented around the first peak, with CT events further subdivided into low energy CT events and high energy CT events that may be used in additional analysis or image generation (e.g., material decomposition).

As indicated above, the X-ray source may be configured to have a low flux. The detector may be configured as a low noise photon counting detector with sub-pixel spatial resolution. Use of one or more low electronic noise ASICs (application specific integrated circuits) may permit collection of partial or shared charges deposited in adjacent pixels due to charge splitting. The center of gravity of such split or shared charges may be calculated in order to reach a sub-pixel spatial resolution, which may be particularly useful for CT image quality. Further, the detector may be configured as a photon counting CZT or other solid state detector configured for detection of both NM and CT photons. In some embodiments, the detector may include pixels having a size of about 0.5 millimeters (other pixel sizes may be employed in other embodiments), and may be configured to detect charge sharing or splitting, and may include or be operably connected to a low noise ASIC configured for charge sharing signal detection and/or sub-pixel calculations.

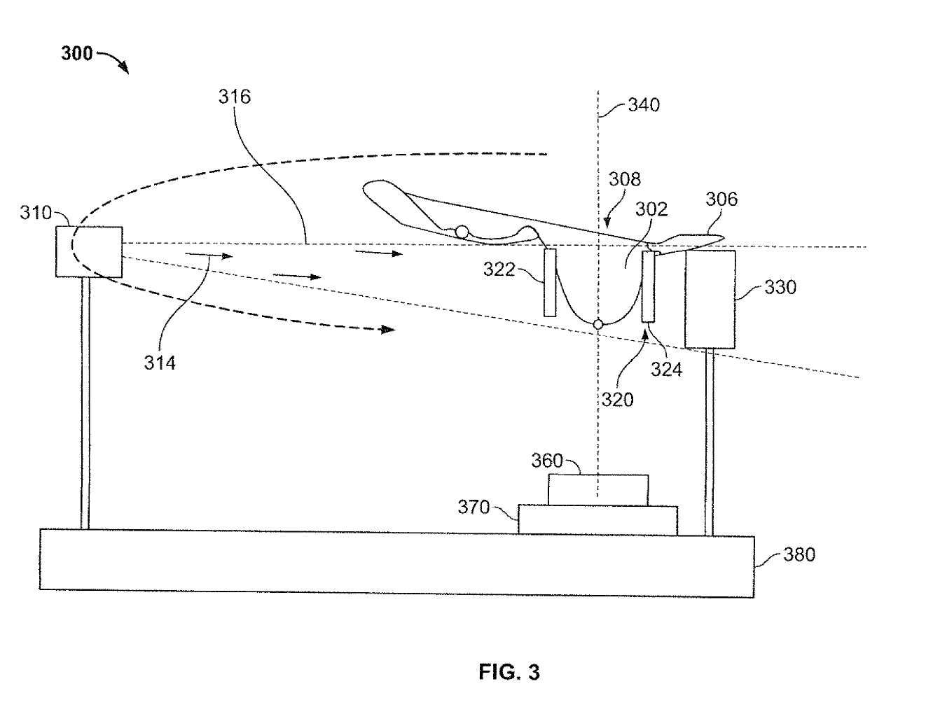

One or more collimators may be employed in conjunction with the detector. For example, a cone beam collimator configured to focus on a focal spot of the X-ray source may be utilized in various embodiments. The x-ray source may be disposed at a relatively large distance from the detector (and collimator) relative to the object being imaged, so that the cone beam collimator may approximate parallel reception of gamma rays originating within the object. In some embodiments, the cone beam collimator may be configured such that a field of view (FOV) of a camera including the cone beam collimator includes locations proximate a chest wall (see FIG. 3). A line of sight between the x-ray source (e.g., x-ray source 310) and an edge of the detector (e.g., detector 330) closest to the patient may be substantially parallel to the chest wall and close to the chest wall, as seen, for example, in FIG. 3.

Further, in various embodiments, a hybrid scanning system may be configured to operate in plural acquisition modes. For example, a hybrid scanning system may be configured to operate in a SPECT/CT mode with the X-ray source and detector rotating around a central axis of an object to be imaged. The X-ray source and detector may be disposed asymmetrically about the central axis with the X-ray source a greater distance from the central axis than the detector. Similarly, a collimator associated with the detector may be asymmetrically disposed, with the collimator closer to the object than a distance from the X-ray source to the object. The acquisition of data may be substantially continuous during the rotation in some embodiments, while in other embodiments data may be acquired intermittently in a “step and shoot” manner. A slow rotation (e.g., about 300 seconds for a single rotation around the object to be imaged) may be employed. As another example, a static or planar acquisition mode may be employed.

In some embodiments, one or more stabilization devices or components may be employed. For example, in an example MBI embodiment, a plastic plate may be used on top of a breast to be imaged for static acquisition. As another example, two plastic plates (one on top of the breast and one on the bottom of the breast) may be employed for SPECT/CT acquisition. In alternate embodiments, different stabilization devices having an appropriately low attenuation factor may be employed.

Further still, in various embodiments, a hybrid scanning system may be configured to have an adjustable or adaptive distance or positioning between the object to be scanned (e.g., the center or a central axis of the object) and the detector (e.g., a face of a collimator used with the detector). For example, in some embodiments, to help maintain the focus of the collimator on the focal spot of the X-ray source without extensive re-focusing, a distance between the detector (e.g., a face of the collimator) and the X-ray source may be maintained constant. However, because the size of objects to be imaged may vary, and because it may be desirable to have the distance between the object to be imaged and the detector to be as small as possible, the system may be configured to allow the detector to be adjustable relative to the object, either by moving the object to the detector or the detector to the object. In embodiments where the X-ray source and detector are rotated about the object, the X-ray source and detector may be adjustable relative to the center of rotation.

Thus, as indicated above, various components or aspects may be particularly adapted or configured for use in a hybrid SPECT/PCT system. One or more of the following aspects of the system may be configured or adapted for use in a hybrid scanning system: The X-ray source may be disposed at a greater distance from the detector than the detector is from the object, allowing approximation of parallel collimation of gamma rays from the object along with cone beam collimation focused on a focal spot of an X-ray source for X-ray reception of CT events. The X-ray source may be configured for low flux to approximate or otherwise correspond to an event rate for NM events. Low noise circuitry and sub-pixel resolution may be employed to provide improved CT image quality. The distance between the object to be imaged and the detector may be adjustable to permit a constant distance between the X-ray source and the detector (and/or collimator).

A technical effect of at least some embodiments provides concurrent or simultaneous acquisition of physiological and anatomical data (e.g., concurrent or simultaneous acquisition of CT and SPECT data). A technical effect of at least some embodiments provides imaging of a portion of a patient (e.g., a breast or organ) in a hybrid photon counting SPECT/CT mode or in planar static acquisition mode without requiring changing the positioning of the portion of the patient. A technical effect of at least some embodiments allows use of the same detector for SPECT and CT. A technical effect of at least some embodiments includes improved registration of images developed using different modalities.

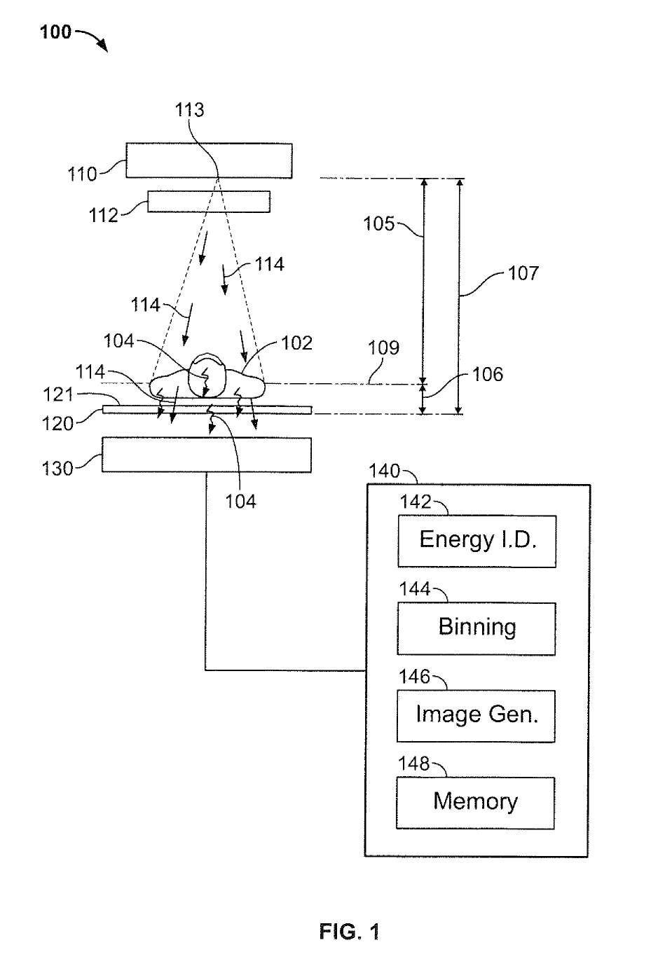

FIG. 1 provides a schematic view of a hybrid scanning system 100 formed in accordance with various embodiments. The depicted system includes a X-ray source 110, a collimator 120, a detector 130, and a processing unit 140. In FIG. 1, the system 100 is configured to obtain CT and SPECT scanning data of the object 102. The object 102 has been administered a radiopharmaceutical, resulting in the emission of gamma rays 104 from the object. The gamma rays 104 may be detected by the detector 130 to obtain SPECT scanning data. Also, the X-ray source 110 emits X-rays that pass through the object 102 and are detected by the detector 130 to obtain CT scanning data. The detector 130 and the processing unit 140 in the illustrated embodiment may simultaneously or concurrently obtain, acquire, and/or process SPECT and CT scanning data. For example, a time period for obtaining SPECT data may overlap partially or entirely with a time period for obtaining CT data, with the detector 130 positioned or oriented in the same position relative to the object 102 while obtaining both the SPECT and CT data. Further, CT events (impingement of X-rays on the detector 130) and SPECT events (impingement of gamma rays on the detector 130) may occur and be detected in a simultaneous or overlapping manner. For example, during a single hybrid scan, an individual X-ray or series of X-rays may be detected by the detector 130 and identified and/or analyzed by the processing unit 140, followed by an individual gamma ray or series of gamma rays that may be detected by the detector 130 and identified and/or analyzed by the processing unit 140, followed by an individual X-ray or series of X-rays detected by the detector 130 and identified and/or analyzed by the processing unit 140, and so on, over the course of the single hybrid scan. It may be noted that, in various embodiments (e.g., systems configured for planar imaging, among others), a position of a gamma ray may be measured directly from pixel location, but the position of an X-ray may be calculated using a group of two or more adjacent pixels to obtain a sub-pixel spatial resolution for higher image quality. For example, relatively small pixels (e.g., about one millimeter or less) may be used in connection with a low-noise ASIC to detect low energy signals resulting from a charge sharing effect between or among adjacent pixels. For an example of imaging regarding shared charge, see U.S. Pat. No. 8,405,038, “Systems and Methods for Providing a Shared Charge in Pixelated Image Detectors,” which is incorporated herein by reference in its entirety.

In the illustrated embodiment, the X-ray source 110 is configured to provide X-rays 114 from a focal spot 113 through a filter 112. The X-ray source 110 may be configured as a tube. The X-ray source 110 and/or filter 112 may be configured to provide X-rays at one or more desired energy levels (e.g., spectra). For example, the X-rays may be configured to have a lower energy than the gamma rays of a radiopharmaceutical administered for the scan, providing for easier differentiation of CT and NM events based on the energies of the events. Additionally, or alternatively, the X-ray source 110 and/or filter 112 may be configured to provide X-rays having a double-peaked energy spectra, allowing for high and low energy X-rays to be separately identified and used, for example, in connection with a material decomposition analysis.

The X-ray source 110 in various embodiments may be configured to provide X-rays at a substantially lower flux than conventionally used for comparable scans of the same body portion or organ. Generally speaking, conventional X-ray sources may provide X-rays at a substantially higher rate than an event rate (e.g., events per pixel per second) from an administered radiopharmaceutical. However, because the CT and NM information may be obtained over corresponding time periods (e.g., identical, substantially the same duration, or overlapping, among others), use of conventional X-ray flux may result in a higher dosage of X-rays than necessary or useful over the duration of the entire hybrid scan. Thus, in various embodiments, the flux of X-rays provided by the X-ray source may be reduced to correspond to the rate of events provided by an administered radiopharmaceutical. For example, the X-ray flux may be reduced so that substantially the same amount of X-rays as gamma rays are received by the detector 130. As other examples, the X-ray flux may be reduced so that about two, three, or five, among others, times as many X-rays as gamma rays are received by the detector 130.

The depicted collimator 120 is configured to permit passage of X-rays 114 and gamma rays 104 at a predetermined angle (or range of angles) and to block other X-rays 114 and gamma rays 104. Thus, the collimator 120 may be configured to permit the directionality of received X-rays 114 and gamma rays 104 by the detector 130 to be known or determined. In various embodiment, the collimator 120 may have bores or channels corresponding to pixels of the detector 130. In the illustrated embodiment, the collimator 120 is configured as a cone beam collimator focused on the focal spot 113 of the X-ray source 110.

It may be desirable, in NM applications, for a collimator to be configured to allow gamma rays to pass through the collimator 120 generally parallel to each other and generally perpendicular to the detector 130. However, if the collimator 120 is focused on the focal spot 113, the collimator 120 may deviate from such a parallel guidance of gamma rays perpendicular to the detector, and the gamma rays impinging upon the detector will not be entirely parallel to each other and will impinge upon the detector at an angle from parallel. The closer the focal spot 113 is to the collimator 120, the more pronounced will be the deviation. In the illustrated embodiment, the deviance from parallel (or deviance from perpendicular to the detector 130) guidance through the collimator 120 is reduced by increasing the distance between the object 102 and the X-ray source 110 relative to the distance between the object 102 and the detector 130 (e.g., the face of the collimator 120 nearest the object 102). Put another way, reducing the distance to the object 102 from the detector 130 or collimator 120 relative to the distance to the object 102 from the X-ray source allows for an approximation of parallel passage of gamma rays 104 from object 102 through collimator 120 to pixels of detector 130 (e.g., substantially perpendicular to receiving faces of pixels), while still maintaining a reasonable or practical distance between the X-ray source 110 and the detector 130 and/or a reasonable detector size.

In the illustrated embodiment, the X-ray source 110 (e.g., the focal spot 113 of the X-ray source) is positioned a total distance 107 from a face 121 of the collimator 120 oriented closest to the object 102. Further, a center 109 of the object 102 is positioned a first distance 105 from the focal spot 113 and a second distance 106 from the face 121 of the collimator 120. Because the first distance 105 and the second distance 106 are substantially different in the illustrated embodiment, the X-ray source 110 and the detector 130 (and/or collimator 120) may be understood as being asymmetric or substantially asymmetric about the center 109. The first distance 105 may be substantially larger than the second distance 106. For example, in various embodiments, the first distance 105 may be about twice the second distance 106, about three times the second distance 106, about five times the second distance 106, or about ten times the second distance 106, among others. In the illustrated embodiment, the first distance 105 may be about 50 centimeters and the second distance 106 may be about 10 centimeters.

Further, the values of the first distance 105 and the second distance 106 may change due to changes in the position of the center 109, while the total distance 107 is maintained generally constant to reduce or minimize re-focusing of the collimator 120. For example, if an object is larger than the object 102, the larger object may be positioned with an edge or border proximate the face 121 of the collimator 120; however, the second distance 106 may increase (and the first distance 105 decrease) because the object is larger. Similarly, while maintaining the total distance 107 constant, the first distance 105 may increase and the second distance 106 decrease if an object smaller than the object 102 is scanned. In various embodiments, the X-ray source 110 and the detector 130 may be fixed to a structure to maintain the total distance 120 generally constant, and the structure may be adjustable relative to the object 102 to allow for the first distance 105 and the second distance 106 to be adjustable, as appropriate for various object sizes. For further discussion of an example embodiment, see FIG. 3 and the related discussion.

Returning to FIG. 1, the depicted detector 130 may be configured as a photon counting detector with a plurality of pixels arranged in an array. The collimator 120 may be affixed or mounted to the detector 130. Generally the detector 130 is configured to receive X-rays from the X-ray source 110 that have passed through the collimator 120 as well as gamma rays from an administered radiopharmaceutical that emanate from the object 102 and have passed through the collimator 120. As shown in FIG. 1, the detector 130 may be disposed in the same position and orientation relative to the object 102 when receiving both the gamma rays 104 and the X-rays 114.

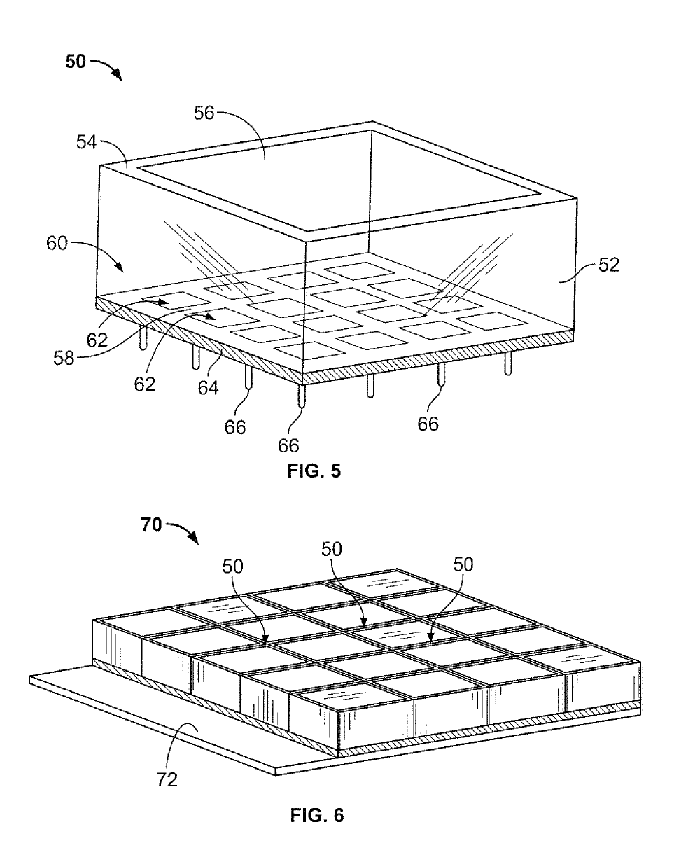

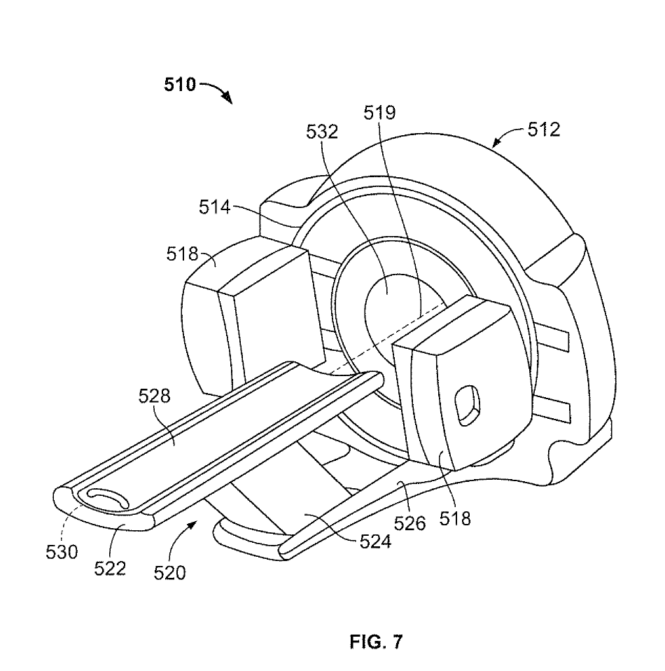

The pixels of the detector 130 may be relatively small, and may be configured for sub-pixel resolution and detection of charge sharing. For example, in some embodiments, the pixels may be about 0.5 millimeters and the detector 130 may provide a resolution of about 0.25 millimeters. Example embodiments of detectors are provided in FIGS. 5 and 6 and the related discussion. The detector 130 is operably connected to the processing unit and provides information to the processing unit 140 regarding detected events, including energy levels of the events and a pixel (or pixels) for which each event was detected. For example, for each event (or shared event), a value corresponding to the energy of the event may be maintained in a peak and hold (P&H) circuit for a pixel (or group of pixels for a shared event). Events may be counted over plural reading cycles, with a running count of events for each pixel used to generate one or more images.

In the illustrated embodiment, the processing unit 140 is operably connected to the detector 130. The depicted processing unit 140 is configured to acquire, obtain, and/or process scanning information from the detector 130 and, based on the scanning information, to identify and/or categorize types of events (e.g., SPECT events, CT events, high energy CT events, low energy CT events, or the like) detected by the detector. Generally, in various embodiments, the processing unit 140 (and/or any sub-unit or module of the processing unit 140) may be understood as a processing circuitry unit and may include processing circuitry such as one or more field programmable gate array (FPGA), application specific integrated circuit (ASIC), integrated circuit (IC), or microprocessor. The processing unit 140 in various embodiments may be configured to execute one or more algorithms to perform functions or operations described herein. The one or more algorithms may include aspects of embodiments disclosed herein, whether or not expressly identified in a flowchart or as a step of a method.

In the illustrated embodiment, the processing unit 140 is configured to obtain information (e.g., energy levels received by one or more pixels) regarding events detected by the detector 140. Based on the energy levels of the events, the processing unit 140 may identify CT information corresponding to CT events and NM information corresponding to NM events. For example, the depicted processing unit 140 identifies and/or categorizes a given event as a CT event or a NM (e.g., SPECT) event based on whether the event has an energy level falling within a NM energy window or one or more CT energy windows (or bins). The processing unit 140, in various embodiments, may be configured to generate a CT image using information identified as corresponding to CT events (e.g., counts of CT events or CT photons received by each pixel) and/or to generate a NM image using information identified as corresponding to NM events (e.g., counts of NM events or NM photons received by each pixel). In the illustrated embodiment, the processing unit includes an energy identification module 142, a binning module 144, an image generation module 146, and a memory 148.

In the illustrated embodiment, the energy identification module 142 is configured to read, identify, or otherwise determine an energy level for events of energy reception by one or more pixels of the detector 130. For example, the energy identification module 142 may read energies of the pixels of the detector 130 (e.g., peak and hold values for each pixel of the detector 130) during a given reading cycle. Further, for pixels for which energy has been received, the energy identification module 142 may be configured to determine if the event is a reception of a photon to be counted or if the event should be discarded without counting (e.g., the event was due to Compton Scattering in the object 102, or the event was attributable to noise, among others). Further still, the energy identification module 142 may be configured to identify shared-charge events, and identify a location for such events (e.g., a location based on a center of gravity or weighted average of the portions of the event shared by adjacent pixels).

The depicted binning module 144 is configured to obtain energy information (e.g., the energy levels identified by the energy identification module 142) and organize or categorize events based on the energy levels. Generally, in various embodiments, CT events are grouped together and NM events are grouped together for further analysis. For example, the binning module 144 may determine, for a given event, if the determined energy of the event falls into a CT energy window or a NM energy window. If the energy falls within the CT energy window, the event is grouped with other events falling within the CT window. If the energy falls within the NM energy window, the event is grouped with other events falling within the NM window. In various embodiments, a running total of counted events tabulated by type (e.g., CT, NM) and pixel (e.g., individual pixel, or location corresponding to a group of pixels for a shared event) may be maintained.

It may be noted that, as indicated herein, sub-pixel resolution for CT imaging may be employed in various embodiments. CT images may have a higher resolution than NM images due to the desirability of viewing fine structures in a CT image. Higher resolution may be obtained by using smaller pixels and/or sub-pixel detection, and/or larger groups of information providing larger statistics and lower noise. NM imaging may provide better contrast, for example between a tumor and tissue, so spatial resolution in NM imaging may not be as high a concern as in CT imaging. In various embodiments, the intensity of the X-ray source is low enough such that the rate of X-ray photons impacting the detector is low enough to allow photon-by-photon processing (including energy determination) without pile-up. Further, energy resolving CT may be employed for tissue classification (e.g., to identify calcifications).

It may further be noted that CT or X-ray energy may be below the energy of NM events. Thus, the energy of scattered X-ray photons may not overlap a NM window or range of energies. However, the energy of scattered NM photons may overlap a X-Ray window or range of energies. In some embodiments, the X-ray power is selected so that the number of scattered NM events in the X-ray window (the number of NM events may be limited by the amount of radiopharmaceutical injected to the patient) is relatively low and results in an acceptable amount of distortion. Further, by turning the X-ray source off (for a relatively short period) during imaging, the NM related counts in the X-ray window or range of energies may be estimated and subtracted.

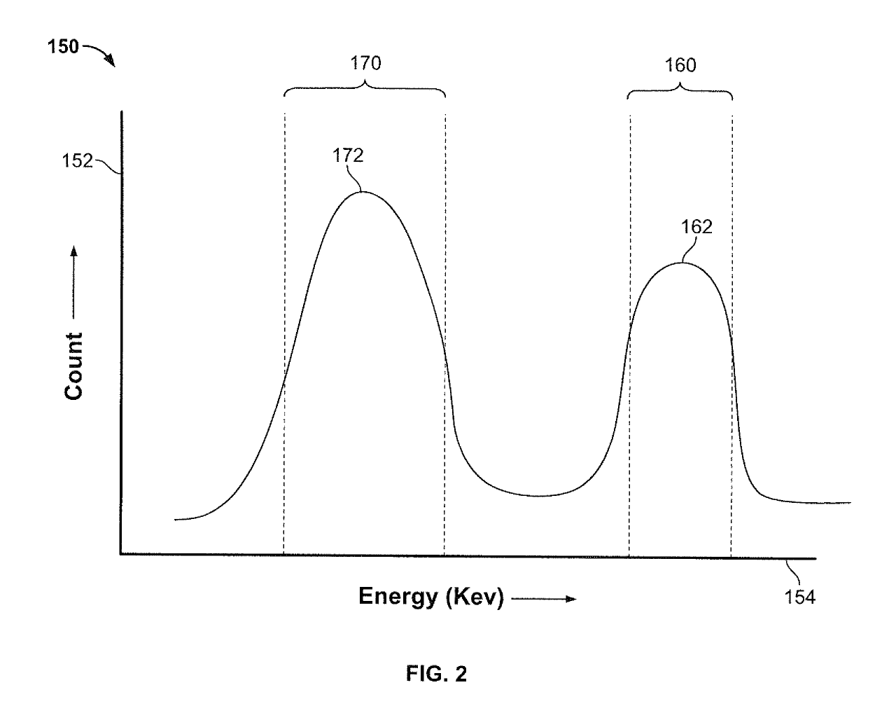

FIG. 2 provides an example graph 150 depicting a distribution of events by energy level in accordance with various embodiments. The graph 150 includes a vertical axis 152 corresponding to a number of events (e.g., incidents of received energy by one or more pixels of a detector) of count and a horizontal axis 154 corresponding to energy level of the events. In the graph 150 depicted in FIG. 2, it may be noted that two peaks (e.g., energy levels for which the number of incidents or events is a local maximum) are shown. The first peak 162 corresponds to the most common energy of NM events. In the illustrated embodiment, the first peak 162 occurs at about 140 KeV. The energy value at which the first peak 162 occurs is determined at least in part by the radiopharmaceutical administered before the scan. The second peak 172 corresponds to the most common energy of CT events. The energy value at which the second peak 172 occurs in the illustrated embodiment is about 60 KeV. The energy value for the second peak 172 is determined at least in part by the configuration and operation of the X-ray source and any associated filters. The energy value of the second peak 172 (and associated energy window) depends upon the voltage of the x-ray tube employed, and may be adjusted by changing the voltage of the x-ray tube. The energy value of the second peak 172 also depends upon the filter (or filters) used, and may be adjusted by changing or adjusting the filter (or filters). Using a broad X-ray band, in conjunction with energy analysis of each detected X-ray photon, may be used to establish tissue energy-dependent absorption properties, which may be used to assist diagnosis, such as determining calcifications which may be associated with a malignancy. Using a narrow energy X-ray beam and energy determination for each X-ray photon may be used for scatter detection and correction, which may improve image quality. In the illustrated embodiment, one NM window is depicted. Some radiopharmaceuticals emit photons at more than one energy. For these isotopes, a plurality of NM energy windows may be defined. Additionally or alternatively, more than one isotope may be used and a plurality of corresponding MN energy windows may be defined.

Each peak of the illustrated embodiment is disposed within an energy window. The width of the energy window may be selected based on one or more statistical determinations. The width of the energy window may be set around a given peak so that the energy window includes as many events corresponding to the same type of event (e.g., CT, NM) without including events of a different type, or including few events of a different type. For example, the width of a window disposed about a peak energy of CT events is preferably configured to include as many CT events as possible while excluding, or minimizing, NM events inside the window. In the illustrated embodiments, a first window 160 or NM window defines an energy band around the first peak 162, and a second window 170 or CT window defines an energy band around the second peak 172. The processing module 140 is configured to identify events having reported or determined energies within the first window 160 as NM events, and to identify events having reported or determined energies within the second window 170 as CT events. Any events that do not fall within one of the energy windows may be discarded, for example, as indeterminate events.

The energy or spectra provided by the X-ray source and/or a radiopharmaceutical may be selected to provide a sufficient distance between a CT peak and a NM peak for the radiopharmaceutical to allow for sufficient differentiation between the borders or edges of the corresponding energy windows (e.g., to prevent overlap between a CT and NM energy window, to provide a minimum indeterminate band between CT and NM energy windows, or the like). Additional or alternative energy windows (or bins) may be employed in alternate embodiments. For example, in some embodiments, the distribution of events by energy may include 3 peaks (a low energy CT peak, a high energy CT peak, and a NM peak), with 3 corresponding windows (a low energy CT window, a high energy CT window, and a NM energy window) employed for grouping events. Use of multiple CT windows or bins may provide for improved material decomposition analysis in some embodiments.

Returning to FIG. 1, the depicted binning module 144 is configured to identify all events having a corresponding energy level (e.g., as determined by the energy identification module 142) in the first window as NM events (e.g., the binning module 144 may record a NM count for the corresponding pixel or location), and identify all events having a corresponding energy level in the second window 170 as CT events (e.g., the binning module 144 may record a CT count for the corresponding pixel or location). In various embodiments, CT events and NM events may be obtained by the detector concurrently (e.g., time periods for collecting or detecting the CT and NM events may overlap entirely, substantially, or partially), and the detected events may be separated, identified, and/or categorized substantially immediately. In various embodiments, the same detector may be used in a similar position and/or orientation to collect CT and NM scanning information concurrently or simultaneously, without re-positioning the detector 130 relative to the object 102, without requiring the use of different detectors for different types of information, without requiring the additional for set-up or scanning using different detectors or scanning positions, or the like.

In the illustrated embodiment, the image generation module 146 is configured to generate one or more images using information acquired by the detector 130 and identified or categorized by one or more aspects of the processing module 140. For example, the image generation module 146 may generate a NM image using NM information including a running total of counted NM events for each location or pixel, with locations or pixels having a higher number of total counts being assigned a higher value or brighter shade of a gray scale (or a brighter or different color) for a corresponding portion of the image. Similarly, the image generation module 146 may generate a CT image using CT information including a running total of counted CT events for each location or pixel, with locations or pixels having a higher number of total counts being assigned a higher value or brighter shade of a gray scale for a corresponding portion of the image. Additionally or alternatively, the image generation module 146 may be configured to generate an overlayed or otherwise combined image using both CT information and NM information. In some embodiments, where CT and NM information were obtained simultaneously or concurrently with the detector 130 in the same position and orientation relative to the object 102 for both types of information, a mechanical, natural, or automatic registration of the images may be obtained, as each group of information may be collected at or about the same time and from about the same physical perspective with respect to the object 102.

FIG. 3 provides a schematic view of a system 300 for hybrid scanning of an object 302 (e.g., breast). The system 300 may include various generally similar aspects as the system 100 discussed above. As shown in FIG. 3, the system 300 is configured for rotation about the object 302 to be scanned, and also provides for adjustment of the distance between the object 302 and a detector 330 and/or collimator, while providing a constant distance between an X-ray source 310 and the detector 330. As seen in FIG. 3, the object 302 includes at least a portion of a torso of a patient, and the detector 330 and X-ray source 310 are configured to be rotated around an axis passing through the object 302, with the axis oriented substantially normal to the torso of the patient. In the embodiment depicted in FIG. 3, the table 306 supports a patient, with the object 302 (portion of patient to be scanned) protruding through an opening 308 in the table 306. The X-ray source 310 emits X-rays 314 through field of view 316. At least some of the X-rays 314 pass through the object 302 and to the detector 330 (e.g., through a collimator (not shown)). It may be noted that a collimator generally similar to the collimator 120 may be included in various embodiments; however, a collimator is not illustrated in FIG. 3 for simplicity and clarity of illustration. A cone beam collimator, for example, may be associated with the detector 330 and interposed between the detector and the object 302. Further, the patient has been administered a radiopharmaceutical previous to the scan, so that gamma rays are emitted from the object 302 to the detector 330. The system 300 also includes a stabilizer assembly 320 joined, mounted, or otherwise affixed to the patient and/or the table 306. The stabilizer assembly 320 is configured to stabilize the object 302 and/or maintain the object 302 in a desired position during a scan. The stabilizer assembly 320 depicted in FIG. 3 includes plates 322 and 324 configured to be disposed on opposite sides of the object 302. Other arrangements of plates or other structures may be employed in alternate embodiments. For example, a concave or “cup” shaped holder may be used. In some embodiments, a cup-shaped holder may be used in conjunction with a suction device configured to urge or draw an object (e.g., breast) into the cup-shaped holder and/or retaining the object in the cup-shaped holder. Differently sized cups may be provided for use with differently sized objects. When cup shaped or cylindrical shaped holders are employed, the rotation of the detector, collimator, and X-ray source may be about a central axis of the holder. The distance between the collimator and the holder may be maintained at a minimal practical amount, with the distance adjusted when the size of the holder is changed to keep the distance between the holder and the collimator at a practical minimum. The plates 322, 324 may be made of a material selected to eliminate, minimize, or reduce attenuation of X-rays and/or gamma rays as the rays pass through the plates 322, 324. In the illustrated embodiment, the plates 322, 324 are configured to hold the object 302 securely to minimize or reduce motion during a scan, but not necessarily to compress the object 302, or to compress the object 302 to a substantial degree.

In FIG. 3, the X-ray source 310 and the detector 330 are configured to rotate around a center of revolution 340 of the object 302. For example, a motor 360 or other rotating device may be affixed to the table 306. The motor 360 may also be affixed to a support structure 380 via an adjustment member 370. The X-ray source 310 and the detector 330 may be fixedly mounted to the support structure 380 in a predetermined relationship (e.g., at a set distance at which a collimator associated with the detector 330 is focused on a focal spot of the X-ray source 310) to each other. In turn, the support structure 380 may be adjustably mounted to the motor 360 via the adjustment member 370, such that the support structure 380 (with the X-ray source 310 and detector 330 mounted thereto) may be adjusted or articulated laterally with respect to the motor (e.g., to the left or to the right as shown in FIG. 3.)

The support structure 380 may be slidably connected to motor 330 via the adjustment member 370, which may include one or more slots tracks, or the like, along with one or more locking or securing features to secure the support structure 380 at a desired position during rotation about the motor 360 and the center of rotation 340. For example, if the object 302 is removed from the table 306, and replaced with a smaller object 302 (e.g., an object centered about the center of rotation 340 but having a smaller diameter than the depicted object 302), it may be desirable to move the detector 330 closer to the new, smaller object (e.g., to reduce a distance gamma rays from the object must travel to reach the detector). Accordingly, a securement feature of the adjustment member 370 may be loosened or released, and the support structure 380 articulated or moved laterally to the left as shown in FIG. 3 to bring the detector 330 closer to the object while maintaining the X-ray source 310 at a constant distance from the detector 330. Thus, the support structure 380 may be adjusted relative to the center of rotation 340 or an axis about which the support structure 380 may be rotated about the motor 360.

Once in the desired position, the support structure 380 may be secured, and the support structure rotated (with the X-ray source 310 and the detector 330 therefor also rotated) about the object 302 during a scan. The rotation may be relatively slow (e.g., about 300 seconds per revolution) in various embodiments. In some embodiments, the rotation may be achieved incrementally, for example in a step-and-shoot fashion. For example, the support structure 380 may be rotated a given angular amount and stopped at a first position, with scanning information collected at the first position. The support structure 380 may then be rotated an additional amount and stopped at a second position, at which additional scanning information is collected, and so on.

Thus, in various embodiments, systems or methods for concurrent CT and NM imaging of a portion of a patient, such as a breast, are provided. For example, a system may include a detector including multiple pixels configured to detect CT events corresponding to X-rays emitted from a X-ray source through an object (e.g., breast) to be imaged, and to detect NM imaging events corresponding to gamma rays emitted from a radiopharmaceutical that has been administered to the patient. The detector may be configured for photon counting detection of the CT events and the NM imaging events. Further, for example as seen in FIG. 3, the detector and the X-ray source may be configured to rotate about the object imaged. In some embodiments the detector and the X-ray source may rotate about an axis that is substantially centered on a breast being imaged, with the axis substantially normal to the torso (e.g., the center of rotation 340 as shown in FIG. 3). Further, a cone beam collimator may be interposed between the detector and the object to be imaged (see, e.g., FIG. 1). The cone beam collimator may be asymmetrically positioned relative to the X-ray source around the axis or center of rotation, with the collimator disposed at a shorter distance from the object to be imaged and the center of rotation than the X-ray source. The collimator may be positioned proximate the object to be imaged and focused on the X-ray source. The cone beam collimator may be positioned such that the cone beam collimator permits imaging locations in the breast near the chest wall (see, e.g., FIG. 3). For example, the collimator and detector may be positioned with an edge of the collimator and/or detector proximate to a support configured to support the chest wall or torso of the patient. The system may also include a processing unit (e.g., a processing unit including at least one processor and at least one memory) to identify CT information corresponding to CT events and NM imaging information corresponding to NM imaging events based on corresponding energy levels of events detected by the detector.

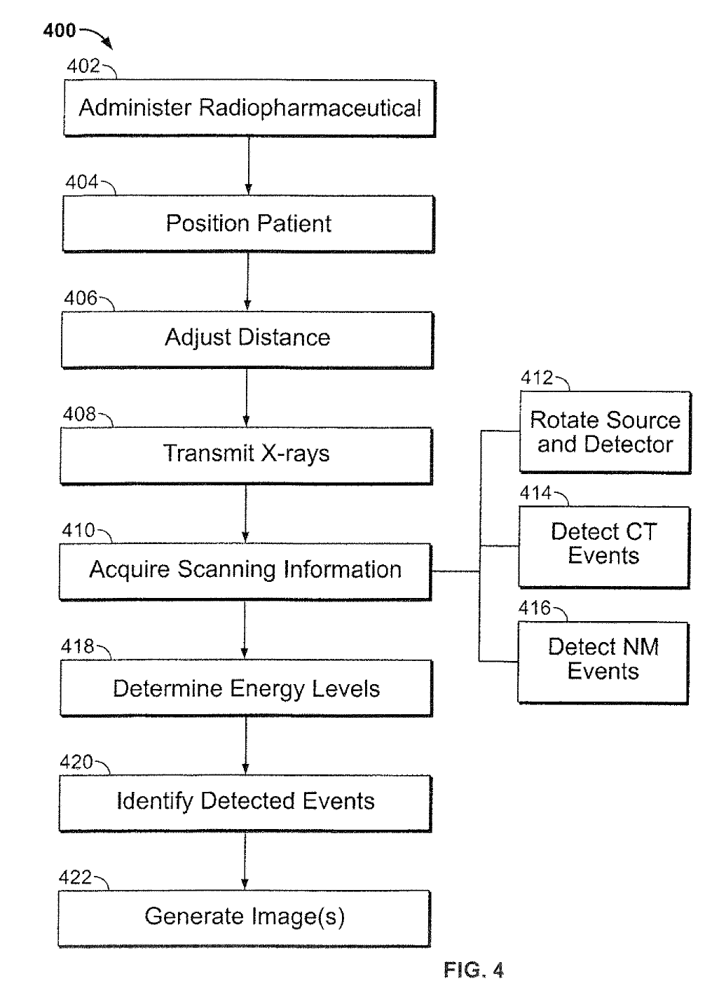

FIG. 4 provides a flowchart of a method 400 for imaging an object (e.g., a portion of a human or animal patient) in accordance with various embodiments. The method 400, for example, may employ or be performed by structures or aspects of various embodiments (e.g., systems and/or methods) discussed herein. In various embodiments, certain steps may be omitted or added, certain steps may be combined, certain steps may be performed simultaneously, certain steps may be performed concurrently, certain steps may be split into multiple steps, certain steps may be performed in a different order, or certain steps or series of steps may be re-performed in an iterative fashion. In various embodiments, portions, aspects, and/or variations of the method 400 may be able to be used as one or more algorithms to direct hardware to perform one or more operations described herein.

At 402, a radiopharmaceutical is administered to a patient. The radiopharmaceutical is configured to emit gamma rays during a subsequent hybrid (e.g., SPECT and CT) scan that may be used to image, for example, physiological and/or anatomical information of the patient. The radiopharmaceutical may be selected to emit gamma rays having an energy distribution that falls in a different band of energy values (e.g., higher energies) than energy associated with X-rays to be used in conjunction with the hybrid scan.

At 404, the patient is positioned. The patient may be placed on a table, for example, with the portion of the patient to be imaged positioned and/or secured in a desired spatial relationship to an X-ray source and a detector, such that X-rays emitted from the source pass through the portion of the patient to be scanned and impinge upon the detector. The detector, for example, may be configured as a pixelated detector configured to receive and detect energy of both gamma rays emitted from the portion of the object to be imaged due to the radiopharmaceutical, as well as X-rays that have passed through the portion of the patient to be imaged.

At 406, a distance from the portion of the patient to be imaged to the detector is adjusted. For example, the detector may be brought as close to the object as reasonably practicable. Generally, in various embodiments, the distance between the detector and the X-ray source may be maintained constant to allow a generally constant focus of a collimator associated with the detector on a focal spot of the X-ray. Further, the distance between the object and the detector (or collimator) may be reduced or minimized relative to the distance between the object and the X-ray source to help minimize the deviation of an angle between the bores or channels of the collimator from a direction generally perpendicular to the detector, or to permit an approximation of parallel passage of gamma rays from the portion of the patient to be imaged through the collimator, while keeping the distance between the X-ray source and the detector at a relatively low distance.

At 408, X-rays are transmitted from the X-ray source. The X-ray source may be configured and controlled, and/or a filter may be used, to help provide a desired direction, amount, and/or energy of X-rays transmitted. For example, the energy of the X-rays transmitted may be selected to provide a substantially different energy level or level(s) compared to energy of gamma rays associated with the radiopharmaceutical administered at 402. As another example, the flux of the X-rays may be controlled so that an amount of X-rays received through the collimator by the detector corresponds to an amount of gamma rays (due to the radiopharmaceutical) received through the collimator by the detector.

At 410, scanning information is acquired via the detector. For example, gamma and X-rays may pass through the collimator and strike or impinge upon the detector, which is configured to detect energy levels of the rays striking the detector, along with locations where the rays strike the detector (e.g., locations may be identified by a particular pixel or pixels that are impinged upon). In various embodiments, the detector be configured for the detection of events shared between pixels and/or for sub-pixel resolution. Generally, the detector may be used to collect information describing events by both energy level of the event (e.g., an energy level represented or reflected by an electric current and/or voltage in the detector). For example, the detector (and/or associated components or circuitry), may be configured to maintain a value corresponding to a peak energy of the event in a P&H of a given pixel or portion of the detector during a reading cycle. The detector may acquire both CT and NM information (e.g., information corresponding to both X-rays and gamma rays) during simultaneous, concurrent, or otherwise overlapping time periods in some embodiments, while the information may be acquired sequentially in other embodiments (e.g., NM information acquired first, followed by CT information). The acquiring of scanning information may be performed in a variety of sub-steps which may be performed simultaneously, concurrently, or in intermittent and/or overlapping fashion in some embodiments. In the illustrated embodiments, the acquiring of scanning information includes steps 412, 414, and 416.

At 412, the X-ray source and detector are rotated about the portion of the patient to be scanned. For example, the X-ray source and detector may be mounted to a support structure that maintains the X-ray source and detector at a fixed distance, with the support structure rotated about a center of rotation. The center of rotation may pass through a central axis of the portion of the patient to be scanned. The support structure may be configured to be adjustable relative to the object and center of rotation to allow for different sized objects to be placed as closely as possible to detector while maintaining a desired distance between the X-ray source and the detector and/or collimator. The rotation may be continuous in some embodiments, and intermittent or in intervals (e.g., step-and-shoot) in other embodiments. The rotation may be relatively slow (e.g., about 300 seconds for a complete revolution about the portion of the patient to be scanned).

At 414 CT events are detected. The CT events correspond to the incidence of X-rays that have passed through the portion of the patient being scanned upon the detector. At 416 NM events are detected. The NM events correspond to the incidence of gamma rays emitted from the portion of the patient being scanned due to the administration of the radiopharmaceutical.

At 418, energy levels of the pixels of the detector are determined. For example, a processing unit may read energy levels in a P&H associated with the detector for each pixel or channel. The energy level read may correspond to an energy level of a single pixel, or a group of two or more adjacent pixels (e.g., in the event of charge-sharing).

At 420 detected events are identified by category or group. For example, CT events may be identified as CT events and grouped (e.g., counted) along with other CT events, while NM events may be identified as NM events and grouped (e.g., counted) along with other NM events. The identification or categorization may be performed based upon the detected energy levels of the respective events. For example, a CT energy window within which CT events are likely to occur may be defined, and a NM window with which NM events are likely to occur may also be defined. Then, for each event with a determined energy falling within the CT energy window, a CT count may be added to a running total for the corresponding pixel or location, and for each event with a determined energy falling within the NM energy window, a NM count may be added to a running total for the corresponding pixel or location. Steps 418 and 420, in various embodiments, may be understood taken together as a combined step or group of steps for identifying CT information and NM information. CT information may include information describing one or more pixel(s) or locations reporting a CT event. NM information may include information describing one or more pixel(s) or locations reporting a NM event. Information, for example, may include a total count of events by type of event for each pixel or location of a detector. For example, a table may be maintained that, for each pixel or location of the detector, maintains a running total of counted CT events and counted NM events.

At 422, one or more images are generated. For example, a CT image may be generated using CT information (e.g., a description of total counts of CT events per pixel or location of the detector). For example, a shade or value of a gray scale may be assigned, based on the count of CT events for a given pixel, to a portion or pixel of a CT image corresponding to the given detector pixel based on total CT counts for the given detector pixel. Additionally or alternatively, a NM image may be generated using NM information in a generally similar fashion. Still further additionally or alternatively, a combined image may be generated. For example, attenuation information from a CT image (or CT information) may be used to correct or adjust a NM image.

Various methods in accordance with embodiments, such as the method 400, may be used in connection with, for example, a pixelated detector 50 as shown in FIG. 5, or, as another example, a sub-pixelated detector as shown in FIG. 5, which may be configured as a sub-pixelated semiconductor photon detector, which in various embodiments is formed from CZT.

It should be noted that the pixelated detectors 50 in various embodiments may be formed from CZT or CdTe. The pixelated detectors 50 include a crystal 52 formed from the semiconductor material. A face 54 of the crystal 52 in some embodiments (as illustrated) includes a single cathode electrode 56. An opposite face 58 of the crystal 52 includes an anode 60 having an array of anode pixels 62. The anode pixels 62 may be of substantially the same size and may be configured as square shaped pixels 62. In various embodiments, the number of anode pixels 62 may be greater or less than the sixteen shown, for example, thirty-two anode pixels 62 may be provided. It also should be noted that the thickness of the crystal 52 may vary between less than one millimeter to several centimeters. In some embodiments, a thickness of several millimeters is used so as to substantially absorb at least a large portion of the impinging photons. Thus, the thickness depends on the energy of the photon to be detected. In operation, a voltage difference applied between the cathode electrode 56 and the anode 60 generates an electric field in the crystal 52.

In operation, when a photon having energy typical of the energies of photons used in SPECT, x-ray, CT or PET applications is incident on the crystal 52, the photon generally interacts with the crystal 52 and pairs of mobile electrons and holes in a small localized region of the crystal 52 are generated through a secondary ionization process. As a result of the applied electrical field, the holes drift to cathode 56 and the electrons drift to anode 60, thereby inducing charges (also referred to as charge clouds or electron clouds) on the anode pixels 62 and the cathode 56. The induced charges on anode pixels 62 are sensed and may be partially preprocessed by appropriate electronic circuits (e.g., application specific integrated circuits (ASICs)) within a detector base 64 and on which the pixelated detector 50 is mounted. For example, a plurality of channels forming a readout amplifier chain may be provided. The detector base 64 includes connection members, for example, connection pins 66 for mounting to a motherboard (not shown) and transmitting signals from the ASICs to the motherboard. Signals from the induced charges on anode pixels 62 are used to determine charge information, including any or all of the time at which a photon is detected, how much energy the detected photon deposited in the crystal and where in the crystal the photon interaction took place as described in more detail herein (e.g., using a row/column summing method). This information may then be used to reconstruct an image as known in the art.

FIG. 6 illustrates a rectangular gamma camera 70 that includes a plurality, for example, twenty pixelated detectors 50 arranged to form a rectangular array of five rows of four detectors 50. The pixelated detectors 50 are shown mounted on a motherboard 72. It should be noted that gamma cameras having larger or smaller arrays of pixelated detectors 50 may be provided. It should also be noted that the energy of a photon detected by a pixelated detector 50 is generally determined from an estimate of the total number of electron-hole pairs produced in the crystal 52 of the detector 50 when the photon interacts with the material of the crystal 52. This count is generally determined from the number of electrons produced in the ionizing event, which is estimated from the charge collected on the anode 60 of the detector 50 using the various embodiments.

If all the electrons and holes produced by a photon detected in the detector 50 are properly collected by the detector electrodes, then the induced charge on either the anode 60 or the cathode 56 of the detector 50 is a correct measure of the energy of the photon. However, the energy response for each pixel, and in particular, the peak position for each peak may shift in the energy spectrum and affect the acquired data used to reconstruct an image. Using the various embodiments, the shifting may be minimized or corrected using a known relationship between the location of the pixels and the anode signals as controlled, for example, by the shaping and connection of the pixels.

The pixelated detectors of the various embodiments may be provided as part of different types of imaging systems, for example, NM imaging systems such as positron emission tomography (PET) imaging systems, SPECT imaging systems and/or x-ray imaging systems and CT imaging systems, among others. For example, FIG. 7 is a perspective view of an exemplary embodiment of a medical imaging system 510 constructed in accordance with various embodiments, which in this embodiment is a SPECT imaging system. The system 510 includes an integrated gantry 512 that further includes a rotor 514 oriented about a gantry central bore 532. The rotor 514 is configured to support one or more NM pixelated cameras 518 (two cameras 518 are shown), such as, but not limited to gamma cameras, SPECT detectors, multi-layer pixelated cameras (e.g., Compton camera) and/or PET detectors. As indicated above, in various embodiments, the medical imaging system 510 also includes an x-ray tube (not shown) for emitting x-ray radiation towards the detectors. In various embodiments, the cameras 518 are formed from pixelated detectors as described in more detail herein. The rotors 514 are further configured to rotate axially about an examination axis 519.

A patient table 520 may include a bed 522 slidingly coupled to a bed support system 524, which may be coupled directly to a floor or may be coupled to the gantry 512 through a base 526 coupled to the gantry 512. The bed 522 may include a stretcher 528 slidingly coupled to an upper surface 530 of the bed 522. The patient table 520 is configured to facilitate ingress and egress of a patient (not shown) into an examination position that is substantially aligned with examination axis 519. During an imaging scan, the patient table 520 may be controlled to move the bed 522 and/or stretcher 528 axially into and out of a bore 532. The operation and control of the imaging system 510 may be performed in any manner known in the art. It should be noted that the various embodiments may be implemented in connection with imaging systems that include rotating gantries or stationary gantries.

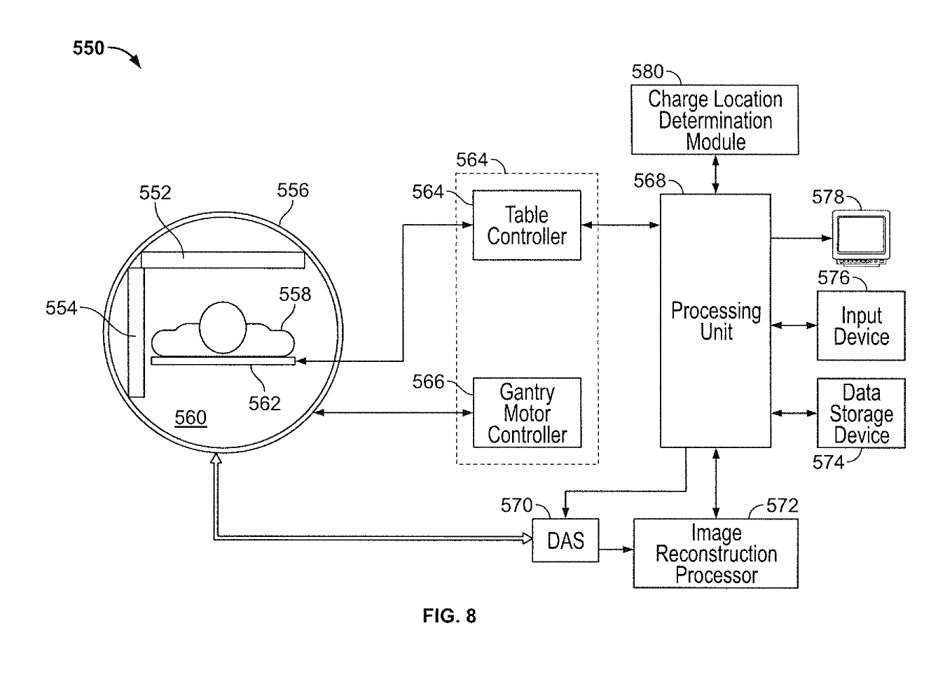

FIG. 8 is a block diagram illustrating an imaging system 550 that has a plurality of pixelated imaging detectors configured in accordance with various embodiments mounted on a gantry. It should be noted that the imaging system may be configured as a hybrid imaging system, such as an NM/CT imaging system. The imaging system 550, illustrated as a SPECT imaging system, generally includes a plurality of pixelated imaging detectors 552 and 554 (two are illustrated) mounted on a gantry 556. It should be noted that additional imaging detectors may be provided. The imaging detectors 552 and 554 are located at multiple positions (e.g., in an L-mode configuration) with respect to a patient 558 in a bore 560 of the gantry 556. The patient 558 is supported on a patient table 562562 such that radiation or imaging data specific to a structure of interest (e.g., the heart) within the patient 558 may be acquired. It should be noted that although the imaging detectors 552 and 554 are configured for movable operation along (or about) the gantry 556, in some imaging systems, imaging detectors are fixedly coupled to the gantry 556 and in a stationary position, for example, in a PET imaging system (e.g., a ring of imaging detectors). It also should be noted that the imaging detectors 552 and 554 may be formed from different materials as described herein and provided in different configurations known in the art.

One or more collimators may be provided in front of the radiation detection face (not shown) of one or more of the imaging detectors 552 and 554. The imaging detectors 552 and 554 acquire a 2D image that may be defined by the x and y location of a pixel and the location of the imaging detectors 552 and 554. The radiation detection face (not shown) is directed towards, for example, the patient 558, which may be a human patient or animal. It should be noted that the gantry 556 may be configured in different shapes, for example, as a “C”, “H” or “L”.

A controller unit 564 may control the movement and positioning of the patient table 562 with respect to the imaging detectors 552 and 554 and the movement and positioning of the imaging detectors 552 and 554 with respect to the patient 558 to position the desired anatomy of the patient 558 within the fields of view (FOVs) of the imaging detectors 552 and 554, which may be performed prior to acquiring an image of the anatomy of interest. The controller unit 564 may have a table controller 564 and a gantry motor controller 566 that each may be automatically commanded by a processing unit 568, manually controlled by an operator, or a combination thereof. The table controller 564 may move the patient table 558 to position the patient 558 relative to the FOV of the imaging detectors 552 and 554. Additionally, or optionally, the imaging detectors 552 and 554 may be moved, positioned or oriented relative to the patient 558 or rotated about the patient 558 under the control of the gantry motor controller 566.

The imaging data may be combined and reconstructed into an image, which may comprise 2D images, a 3D volume or a 3D volume over time (4D).

A Data Acquisition System (DAS) 570 receives analog and/or digital electrical signal data produced by the imaging detectors 552 and 554 and decodes the data for subsequent processing as described in more detail herein. An image reconstruction processor 572 receives the data from the DAS 570 and reconstructs an image using any reconstruction process known in the art. A data storage device 574 may be provided to store data from the DAS 570 or reconstructed image data. An input device 576 also may be provided to receive user inputs and a display 578 may be provided to display reconstructed images.

Moreover, a charge location determination module 580 may be provided to determine a location of a charge or a charge cloud generated by photon (e.g., emission gamma photon or transmission x-ray photons). The charge location determination module 580 may be implemented in software, hardware or a combination thereof.

For an example of a multi-modality detector (e.g., NM and X-ray), see U.S. Pat. No. 7,332,724, “Method and Apparatus for Acquiring Radiation Data,” which is hereby incorporated by reference in its entirety.

It should be noted that the particular arrangement of components (e.g., the number, types, placement, or the like) of the illustrated embodiments may be modified in various alternate embodiments. In various embodiments, different numbers of a given module or unit may be employed, a different type or types of a given module or unit may be employed, a number of modules or units (or aspects thereof) may be combined, a given module or unit may be divided into plural modules (or sub-modules) or units (or sub-units), a given module or unit may be added, or a given module or unit may be omitted.

It should be noted that the various embodiments may be implemented in hardware, software or a combination thereof. The various embodiments and/or components, for example, the modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor further may include a storage device, which may be a hard disk drive or a removable storage drive such as a solid state drive, optical drive, and the like. The storage device may also be other similar means for loading computer programs or other instructions into the computer or processor.

As used herein, the term “computer,” “controller,” and “module” may each include any processor-based or microprocessor-based system including systems using microcontrollers, reduced instruction set computers (RISC), application specific integrated circuits (ASICs), logic circuits, GPUs, FPGAs, and any other circuit or processor capable of executing the functions described herein. The above examples are exemplary only, and are thus not intended to limit in any way the definition and/or meaning of the term “module” or “computer.”

The computer, module, or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer, module, or processor as a processing machine to perform specific operations such as the methods and processes of the various embodiments described and/or illustrated herein. The set of instructions may be in the form of a software program. The software may be in various forms such as system software or application software and which may be embodied as a tangible and non-transitory computer readable medium. Further, the software may be in the form of a collection of separate programs or modules, a program module within a larger program or a portion of a program module. The software also may include modular programming in the form of object-oriented programming. The processing of input data by the processing machine may be in response to operator commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program. The individual components of the various embodiments may be virtualized and hosted by a cloud type computational environment, for example to allow for dynamic allocation of computational power, without requiring the user concerning the location, configuration, and/or specific hardware of the computer system

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the invention without departing from their scope. While the dimensions and types of materials described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112, sixth paragraph, unless and until such claim limitations expressly use the phrase “means for” followed by a statement of function void of further structure.

This written description uses examples to disclose the various embodiments of the invention, and also to enable any person skilled in the art to practice the various embodiments of the invention, including making and using any devices or systems and performing any incorporated methods. The patentable scope of the various embodiments of the invention is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

What is claimed is:

1. A system comprising:
a detector comprising multiple pixels configured to detect computed tomography (CT) events and nuclear medicine (NM) imaging events, the CT events corresponding to X-rays emitted from a X-ray source through an object to be imaged, the NM imaging events corresponding to gamma rays emitted from a radiopharmaceutical that has been administered to the object, the detector configured for photon counting detection of the CT events and the NM imaging events, wherein the detector is configured to detect the CT events and the NM events concurrently, wherein the detector and the X-ray source are configured to rotate about the object to be imaged;
an asymmetrically disposed cone beam collimator positioned proximate the object and configured to be focused on the X-ray source, wherein the asymmetric cone beam collimator is disposed at a shorter distance from the object than a distance from the X-ray source to the object; and
a processing unit comprising at least one processor and at least one memory comprising a tangible and non-transitory computer readable storage medium, the processing module configured to, based on corresponding energy levels of the CT events and the NM imaging events, identify CT information corresponding to the CT events and identify NM information corresponding to the NM imaging events.

2. The system of claim 1, wherein the processing unit is configured to generate a CT image using the CT information, and to generate a NM image using the NM information.

3. The system of claim 1, wherein the X-ray source is configured to be disposed at a first distance from the object and the detector is configured to be disposed at a second distance from the object during scanning, wherein the first distance is substantially greater than the second distance.

4. The system of claim 3, wherein the first distance is between about 3 times to about 5 times greater than the second distance.

5. The system of claim 3, wherein the system further comprises a mounting structure configured to maintain the X-ray source and the detector at a constant distance, wherein the mounting structure is adjustably positionable relative to the object to be imaged.

6. The system of claim 1, wherein the object comprises at least a portion of a torso of a patient, and wherein the detector and X-ray source are configured to be rotated around an axis passing through the object and oriented substantially no/mai to the torso of the patient.

7. The system of claim 1, wherein the cone beam collimator is positioned at a distance from the object and a distance from the X-ray source on which the cone beam collimator is focused wherein parallel reception of gamma rays originating within the object is approximated.

8. The system of claim 1, wherein the object is a breast of a patient, and wherein the detector and the X-ray source are configured to rotate around an axis passing through the breast.

9. The system of claim 1, wherein the processing unit is configured to identify events having an energy within a first energy window as the NM events and to identify events having an energy within a second energy window as the CT events.

10. The system of claim 1, wherein the processing unit is configured to identify plural groups of CT events based on the energy levels of the CT events.

11. A method comprising:
detecting, concurrently, with a detector comprising multiple pixels, computed tomography (CT) events and nuclear medicine (NM) imaging events, the CT events corresponding to X-rays emitted from a X-ray source through an object to be imaged, the NM imaging events corresponding to gamma rays emitted from a radiopharmaceutical that has been administered to the object;
rotating the detector, X-ray source, and a cone beam collimator around the object being imaged during the detecting, wherein the cone beam collimator is disposed proximate the object and associated with the detector, wherein the cone beam collimator is asymmetrically disposed at a shorter distance from the object than a distance from the X-ray source to the object;
identifying, based on energy levels of the CT events, CT information corresponding to the CT events; and
identifying, based on energy levels of the NM imaging events, NM information corresponding to the NM imaging events.

12. The method of claim 11, further comprising generating a CT image using the CT information, and generating a NM image using the NM information.

13. The method of claim 11, wherein the X-ray source is configured to be disposed at a first distance from the object and the detector is configured to be disposed at a second distance from the object during scanning, wherein the first distance is substantially greater than the second distance.

14. The method of claim 11, wherein the cone beam collimator is positioned at a distance from the object and a distance from the X-ray source on which the cone beam collimator is focused wherein parallel reception of gamma rays originating within the object is approximated.

15. The method of claim 11, further comprising adjustably positioning a mounting structure configured to maintain the X-ray source and the detector at a constant distance, wherein the mounting structure is laterally adjusted relative to a central axis of the object to be imaged.

16. The method of claim 11, wherein the object to be imaged is a breast of a patient, further comprising rotating the detector, X-ray source, and cone beam collimator around an axis passing through the breast.

17. A tangible and non-transitory computer readable medium comprising one or more computer software modules configured to direct one or more processors to:
obtain, concurrently, from a detector comprising multiple pixels, imaging information corresponding to computed tomography (CT) events and nuclear medicine (NM) imaging events, the CT events corresponding to X-rays emitted from a X-ray source through an object to be imaged, the NM imaging events corresponding to gamma rays emitted from a radiopharmaceutical that has been administered to the object, wherein the detector, X-ray source, and a cone beam collimator are rotated around the object being imaged during detection of the CT events and the NM imaging events, wherein the cone beam collimator is disposed proximate the object and associated with the detector, wherein the cone beam collimator is asymmetrically disposed at a shorter distance from the object than a distance from the X-ray source to the object;
identify, based on energy levels of the CT events, CT information corresponding to the CT events; and
identify, based on energy levels of the NM imaging events, NM information corresponding to the NM imaging events.

18. The tangible and non-transitory computer readable medium of claim 17, wherein the computer readable medium is further configured to direct the one or more processors to generate a CT image using the CT information and to generate a NM image using the NM information.

19. The tangible and non-transitory computer readable medium of claim 17, wherein the X-ray source is configured to be disposed at a first distance from the object and the detector is configured to be disposed at a second distance from the object during scanning, wherein the first distance is substantially greater than the second distance.

20. The tangible and non-transitory computer readable medium of claim 17, wherein the object to be imaged is a breast of a patient, wherein the detector, X-ray source, and cone beam collimator are rotated around an axis passing through the breast.
